(19) United States
(12) Patent Application Publication
(10) Pub. No.: US 2021/0106470 A1
(43) Pub. Date: Apr. 15, 2021

(54) DYNAMIC COMPRESSION GARMENT AND USES THEREOF

(71) Applicant: Ramin Shahidi, Los Altos, CA (US)

(72) Inventor: Ramin Shahidi, Los Altos, CA (US)

(21) Appl. No.: 17/069,723

(22) Filed: Oct. 13, 2020

Related U.S. Application Data
(60) Provisional application No. 62/915,621, filed on Oct. 15, 2019.

(51) Int. Cl. A61F 13/08; A61H 9/00; A61H 7/00; G16H 50/30; D03D 1/00; D03D 7/00

(52) U.S. Cl. CPC A61F 13/085; D03D 2700/0174; A61H 7/001; G16H 50/30; D03D 1/0088; D03D 7/00; A61H 2201/5007; A61H 2201/1207; A61H 2230/305; A61H 2209/00; A61H 2201/0184; A61H 2201/0176; D10B 2509/00; D10B 2507/00; A61H 9/0092

(57) ABSTRACT

The subject matter presented herein provides an efficacious system and method for diagnosing orthostatic intolerance and for treating orthostatic hypotension. A dynamic compression garment that comprises a servo-controlled splanchnic venous compression with automated binder system is used to regulate blood flow, for instance, during orthostasis. The system includes a programmable controller and a computing device in communication with the controller, an inflator, one or more sensors, and a power supply. The system can collect a user's biometric data, which may be transmitted to the user, a physician in charge of the user, or a third-party. Moreover, the biometric data may be incorporated into a machine learning model that can be used to further program the controller.

### CROSS-REFERENCE TO RELATED APPLICATIONS

This application claims the benefit of U.S. Patent Application No. 62/915,621, filed Oct. 15, 2019, the entire disclosure of which is hereby incorporated herein by reference.

### BACKGROUND OF THE INVENTION

Orthostatic intolerance (OI) is a condition that is characterized by a severe drop in blood pressure upon changing body posture rapidly, such as standing up from a seated position. Orthostatic intolerance can be acute or chronic, and can be considered a subcategory of dysautonomia. One mechanism that contributes to OI is a pooling of blood in lower limbs, after which the body struggles to properly return blood to the heart. This can be simulated in healthy individuals though a head-up tilt test, which suspends a person in a vertical position without allowing them to make use of normal mechanisms to augment venous return, such as a muscle pump. This simulation causes a pooling of blood in the venous system of lower extremities, sequestering about 500-600 mL of blood away from an upper body. The blood redistribution results in a decrease in blood return to the heart, subsequently causing a decrease in stroke volume, cardiac output, and ultimately blood pressure. The body will attempt to compensate by increasing heart rate and blood pressure through a baroreceptor reflex. In healthy individuals, the cardiovascular system would be able to do this for an extended period of time before experiencing syncopal symptoms. However, individuals with OI rapidly experience syncopal symptoms as their body cannot compensate appropriately, resulting in orthostatic hypotension. The effects of orthostatic hypotension are mainly age-dependent and may include high rate of bone loss and muscle degeneration.

Usually, orthostatic hypotension is caused by extended periods of standing or sitting, or by sudden changes of position from sitting to standing or a lying to sitting position. However, orthostatic hypotension can happen in other situations. For example, pilots who experience high-accelerated velocities, e.g., fighter pilots experiencing over 2 g accelerations are prone to adverse effects of orthostatic hypotension as a result of the sudden accelerations. Orthostatic hypotension is a common condition that affects 6 percent of the population, and OI can be difficult to diagnose. Accordingly, there is a need to treat OI.

### SUMMARY OF THE INVENTION

The present disclosure contemplates various aspects of a dynamic compression garment.

In some aspects, the compression garment is used to detect orthostatic intolerance (OI) and/or treat orthostatic hypotension. Aspects of the present disclosure are best understood from the following detailed description when read with the accompanying figures. In accordance with the standard practice in the industry, various features are not drawn to scale.

Herein, a dynamic compression garment, i.e., a servo-controlled splanchnic venous compression with inflatable automated binder system is described. In some embodiments, the system includes a controller and a computing device in communication with the controller. In some embodiments, the system further includes a pumping device, a compression unit, one or more sensors, a valve, and a power supply. The pumping device may be in communication with the controller and the compression unit. The one or more sensors may be in contact with a user to detect biometric data from the user and transmit the biometric data to the controller.

In some embodiments, the system includes controller, a computing device in communication with the controller, an electric signal generator, one or more sensors, and a power supply. The electric signal generator is in communication with the controller and the one or more sensors. The one or more sensors are further in contact with a user's body to detect biometric data from the user and transmit the biometric data to the controller.

In some embodiments, the controller receives cardiovascular data from the user. The controller may receive the biometric data through the one or more sensors. The biometric data may be blood pressure of the user measured by the one or more sensors. In some embodiments, the measured blood pressure is compared to a first and a second predetermined thresholds. The first predetermined threshold may be an upper limit for the blood pressure. Determination of the first and the second predetermined thresholds can be based on the user's health condition, age, gender, etc. The first and the second predetermined thresholds can be changed at any time and added to the controller to reprogram the controller. Upon determination that the measured blood pressure is above the first predetermined threshold, a warning is sent to the user. In some embodiments the warning is sent to at least one of: the user, a physician in charge of the user, and a third-party. Upon sending the warning, the controller stops the process and the binder releases. The second predetermined threshold may be a lower limit for the blood pressure. Upon determination that the measured blood pressure is below the first predetermined threshold, the controller transmits a signal to the electric signal generator. Subsequently, the electric signal generator transmits an electric current to the compression garment which causes the compression garment to become pressurized, i.e., inflated, to increase the blood pressure of the user.

In some embodiments, the inflatable binder is an inflatable abdominal binder to be used around user's abdomen. In some embodiments, the inflatable binder is only activated when the user is standing. In some embodiments, the controller upon determination of a blood pressure below the second predetermined threshold, automatically commands the inflatable binder to inflate. The controller may be programmed to measure the blood pressure at certain times where the blood pressure was previously determined below the second predetermined threshold. The controller may be programmed to automatically repeat the measurement for a certain duration of time.

The system measures the user's blood pressure intermittently and at predetermined times. In some embodiments, the controller is programmable to operate at certain times. The controller may further be programmed to measure the user's blood pressure less frequently during the night while the user is asleep.

In some embodiments, the system may include additional sensors to detect user's biometric data other than the blood pressure, as a second biometric factor. This second biometric factor may be used to determine the user's current physical state, e.g., whether the user is asleep, awake, standing, walking, etc. Upon determination that the second biometric factor deviates from a presumed value, the controller may activate the process of inflating the binder. Some non-limiting examples of second biometric factors include directional movement, pressure, breathing frequency, blood oxygen, blood glucose, temperature, heart rate, and the like.

In some embodiments, a machine learning model is used to program the controller. A suitable machine learning model may be initially trained by a suitable method. The trained machine learning model may use ongoing data of the user's blood pressure for further training and predict the user's blood pressure in future events. In some embodiments, the machine learning model can determine whether or not the user is standing. The machine learning model may measure the blood pressure at certain times where the blood pressure was previously determined below the second predetermined threshold.

In some embodiments, the machine learning model uses user's low blood pressure times to predict when the controller should measure the blood pressure more frequently. Alternatively, the machine learning model uses user's normal or relatively high blood pressure times to predict when the controller should measure the blood pressure less frequently.

In some embodiments, the system includes a machine learning model and one or more additional sensors. The machine learning model may use additional biometric data or other user data detected by the one or more additional sensors to predict required frequency of blood pressure measurements at different times of a day. By way of non-limiting examples, the one or more sensors may be an accelerometer, a gyroscope, a photosensor, a heart-rate sensor, an electrocardiograph sensor, a respiration sensor, an oxygen sensor, a glucose sensor, a pressure sensor, a temperature sensor, and/or a multi-variable sensor. In some embodiments, the controller activates the measurement process upon a pressing of a button. In some embodiments, the controller measures the blood pressure while the inflation process is taking place.

The present disclosure provides several advantages over conventional products and methods. One such advantage can be the use of stimulus-driven contracting fabrics for the detection and treatment of hypotension, including in OI patients. Another advantage of the present disclosure involves the use of a feedback mechanism to actuate contraction. Moreover, data can be detected and stored via system sensors which can contemplate many uses, such as providing a detailed medical history of a patient for the patient's physician and/or to supplement a patient's medical record.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 exemplifies a non-limiting example of a dynamic compression garment comprising a servo-controlled splanchnic venous compression system with inflatable automated binder, in accordance with some embodiments.

FIG. 2 exemplifies a non-limiting example a dynamic compression garment comprising a servo-controlled splanchnic venous compression system with inflatable automated binder, in accordance with some embodiments.

FIGS. 3A-3B are non-limiting flowchart representations of methods of servo-controlled splanchnic venous compression with inflatable automated binder, in accordance with some embodiments.

FIG. 4 exemplifies a non-limiting example of a dynamic compression garment comprising a servo-controlled splanchnic venous compression system with inflatable automated binder, in accordance with some embodiments.

### DETAILED DESCRIPTION OF THE INVENTION

The following disclosure provides several different embodiments, or examples, for implementing different features of the provided subject matter. Specific examples of components and features are described below to simplify the present disclosure. These are, of course, merely examples and are not intended to be limiting. For example, the formation of a first feature over or on a second feature in the description that follows may include embodiments in which the first and second features are formed in direct contact and may also include embodiments in which additional features may be formed between the first and second features, such that the first and second features may not be in direct contact. In addition, the present disclosure may repeat reference numerals or letters in the various examples. This repetition is for the purpose of simplicity and clarity and does not in itself dictate a relationship between the various embodiments or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation illustrated in the figures. The apparatus may be otherwise oriented (rotated 90 degrees or at other orientations) and the spatially relative descriptors used herein may likewise be interpreted accordingly.

The use of compression garments can be efficacious in counteracting the cardiovascular effects of head-up tilt, including preventing syncope, increased heart rate, decreased cardiac output, and blood pressure. One use case for the compression garments is for patients that have altered fluid distribution and thus require external forces to properly distribute blood and extravascular fluid. Another use case is for pilots of fighter jets and other ultra-high-speed planes that experience high accelerations. These two classes of users, amongst others, are prone to a condition called “orthostatic hypotension” (OH).

Although splanchnic venous pooling is a major hemodynamic determinant of OH, it is not specifically targeted by pressor agents, the mainstay of treatment. Additionally, current compression stockings are uncomfortable and are particularly difficult to put on and take off, resulting in poor adherence and, subsequently, deterioration of health status of the user. Blood pressure responses to compression garments during tilt or to prevent orthostatic hypotension are also mixed.

One aspect of the subject matter disclosed herein is a dynamic compression garment, i.e., servo-controlled splanchnic venous compression with inflatable automated binder system. The servo-controlled splanchnic venous compression with inflatable automated binder system specifically targets venous splanchnic circulation. An inflatable binder can be activated manually or with an automated posture detector. The inflatable binder may be activated on an as-needed basis, e.g., when the user is standing. The servo controller ensures a predefined sustained compression level. Thus, the servo controller does not worsen supine hypertension, which is a common comorbidity of other treatment options. Effects of using this system is an immediate onset and offset, compared with short-acting pressor agents that have a peak effect of 1-hour and last several hours. In some embodiments, the servo-controlled splanchnic venous compression with inflatable automated binder system can be combined with pressor agents to produce a greater effect on upright blood pressure. In some embodiments, the servo-controlled splanchnic venous compression with inflatable automated binder system relies less on user's compliance or their ability to apply an effective compression level.

In general, compression of venous capacitance beds with lower body compression garments improves venous return and can be useful in treating OH. The main advantages of this approach are that immediate pressor responses could be achieved on demand and when required, i.e., while standing. Further, the compression garment can be safely combined with virtually any drug therapy. Nonetheless, current compression garments' compliance is low because they are difficult to use at compression levels that are effective. To tackle this issue, disclosed is an automated inflatable binder that can be selectively activated in the upright, or inclined, posture to produce a sustained compression level of 40 mm Hg, i.e., a level known to produce compression of the venous splanchnic circulation. The present application discloses a servo-controlled splanchnic venous compression with an inflatable binder system, which is as effective as midodrine, the current standard of care, in improving orthostatic tolerance and reducing orthostatic symptoms in patients with primary autonomic failure and users who are prone to sudden low blood pressure levels such as jet pilots.

Specifically, the servo-controlled splanchnic venous compression with inflatable automated binder system allows for switching on and off the compression such that one may put on and take off the garments with minimal friction. Should the inflatable automated binder, i.e., the garment, become uncomfortable it is easy to deactivate the garment or turn down the magnitude of compression. In some embodiments, various manual controls are available to the user. As a non-limiting example, the user has access to manual controls to start blood pressure measurement process immediately. As another non-limiting example, the user has access to manual controls to start inflating/deflating the binder immediately. As yet another non-limiting example, the user has access to manual controls to call for help, e.g., via communication systems, immediately.

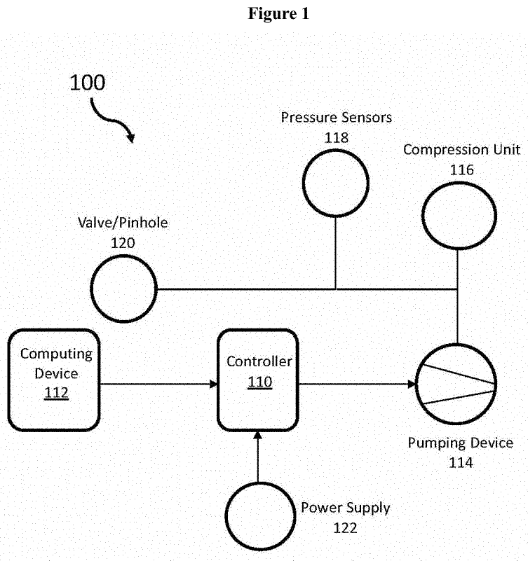

Referring to FIG. 1, the servo-controlled splanchnic venous compression with inflatable automated binder system 100 includes a controller 110 and a computing device 112 in communication with the controller 110, according to some embodiments. In some embodiments, the servo-controlled splanchnic venous compression with inflatable automated binder system 100 includes a pumping device 114, a compression unit 116, one or more pressure sensors 118, a valve 120 and a power supply 122. The pumping device 114 is in communication with the controller 110 and the compression unit 116. The one or more pressure sensors 118 are in contact with a user, e.g., a patient suffering OH or a pilot, to detect biometric data from the user and transmit the biometric data to the controller 110. In some embodiments, a pinhole is used instead of the valve 120. The supply power 122 by way of non-limiting examples may be a battery, a rechargeable battery, a DC source of power, an AC source of power, or any other suitable source of power. The power supply 122 provides continuous operation of the controller 110. In some embodiments, the pumping device 114 and the compression unit 116 are integrated into a single unit.

In some embodiments, an abdominal binder is used as the inflatable binder in the servo-controlled splanchnic venous compression with inflatable automated binder system 100. In some embodiments, other types of binders are used as the inflatable binder in the servo-controlled splanchnic venous compression with inflatable automated binder system 100. According to some embodiments, an inflatable abdominal binder is developed from components that apply a sustained servo-controlled compression pressure. The compression can be programmable to apply various ranges of pressures. By way of non-limiting examples, the pressure ranges can be from 10 to 50 mm Hg, 10 to 40 mm Hg, 10 to 30 mm Hg, 20 to 50 mm Hg, 20 to 40 mm Hg, or 20 to 30 mm Hg. In some embodiments, the compression system consists of a commercially available abdominal band or lumbar support garment made of polyester cloth with adjustable Velcro, and an inflatable cuff, e.g., a commercially available blood pressure cuff, placed underneath. The binder can attach to the user's body around the abdomen area with an inflatable binder placed at a level of umbilicus. The inflatable binder can be pressurized by the compression unit 116/pumping device 114 (collectively, “an automated inflator”). The automated inflator can monitor and maintain a compression level by inflating or deflating the binder. In some embodiments, a commercial inflator and an air pump are used. The pumping device 114 can be located in a cart next to the user. In some embodiments, a portable automated pumping device and controller box is developed that is attached to the binder. Regardless of the type, the inflator can be manually activated to provide a required compression level, e.g., of about 40 mm Hg, for a certain period of time, e.g., less than two minutes, before a post-intervention orthostatic tolerance test. Inflation pressure can be maintained constant by a servo-controlled circuit. A time taken to inflate or deflate the binder with either pump may be less than 1 minute, preferably less than 30 seconds.

In some embodiments, the servo-controlled splanchnic venous compression with inflatable automated binder system is used in combination with midodrine or droxidopa, known standards of care. Midodrine and droxidopa are current approved drugs for the treatment of neurogenic OH. Midodrine and droxidopa are pro-drugs that are activated through their conversion to desglymidodrine and norepinephrine, respectively. Desglymidodrine is a selective α1-adrenoreceptor agonist. Midodrine and other current pressor agents can induce or worsen supine hypertension, given that both drugs increase both supine and standing blood pressure. Thus, the OH, which is a difference between supine and standing blood pressure, is often not selectively improved. Therefore, it is less likely that these drugs help reduce the risk of renal disease, heart failure, and overall mortality associated with OH, and they may be contraindicated in patients with significant cardiovascular disease. Moreover, side effects of these drugs, e.g., urinary retention and piloerection for midodrine, exaggerated fluid retention and hypokalemia for fludrocortisone, further limit their use. Finally, direct vasoconstrictors such as midodrine do not specifically target a major hemodynamic mechanism responsible for neurogenic OH, the reduction in venous return because of failure of sympathetically mediated contraction of capacitance beds.

In some embodiments, certain fabrics capable of contracting are used. Such fabrics can contract upon the application of certain stimuli, e.g., electric stimuli. In some embodiments, an electrical current is used to contract the binder. In other embodiments, a magnetic field is used to contract the binder. For example, an electron induced biopolymer can be used as the binder. As another example, a memory material can be used as the binder. The memory material may be a memory metal, memory alloy, smart metal, smart alloy, or muscle wire. Such memory or smart metals and allows can be made from combinations of, by way of non-limiting examples, copper, aluminum, nickel, titanium, zinc, and iron. For example, two common commercially available memory metals are copper-aluminum-nickel and nickel-titanium. The electrical current causes the contracting binder to contract, resulting in increasing the user's blood pressure. In some embodiments, a string- or a cable-pulling mechanism can be used for inflation, i.e., contraction. In such embodiments, the electrical current causes a wire, or a string, to be pulled tight. The mechanism would be similar to a motor pulling together a string, or to a coil pulling a spring or wire, in which the string- or cable-pulling tightens the fabric or the sleeve.

In some embodiments, one or more sensors are used to detect data from the user. By way of non-limiting examples, the one or more sensors may be an accelerometer, a gyroscope, a photosensor, a heart-rate sensor, an electrocardiograph sensor, a respiration sensor, an oxygen sensor, a glucose sensor, a pressure sensor, a temperature sensor, and/or a multi-variable sensor. In some embodiments, the one or more sensors may include more than one of a same type of sensor, e.g., two or more accelerometers. In some embodiments, one or more ancillary communication systems, e.g., Bluetooth, Wi-Fi, mobile phone interface, computer interface, GPS, can be used.

In some embodiments, the controller 110 receives cardiovascular data from the user. The controller 110 may receive biometric data through the one or more pressure sensors 118. The biometric data may be blood pressure of the user. In some embodiments, the measured blood pressure is compared to a first predetermined threshold. The first predetermined threshold may be an upper limit for the blood pressure, e.g., 100 mm Hg, 110 mm Hg, 120 mm Hg, 130 mm Hg, 140 mm Hg, 150 mm Hg, 160 mm Hg, 170 mm Hg, 180 mm Hg, 190 mm Hg, or 200 mm Hg. Determination of the first predetermined threshold can be based on the user's health condition, age, gender, etc. The first predetermined threshold can be changed at any time and added to the controller 110 to re-program the controller 110. Upon determination that the measured blood pressure is above the first predetermined threshold, a warning is sent to the user. In some embodiments the warning is sent to at least one of: the user, a physician in charge of the user, and a third-party. Upon sending the warning, the controller 110 stops the process and the binder releases. In some embodiments, the controller 110 may receive non-biometric data. For example, the data received include positional data, e.g., position of the user, acceleration of the movement of the user, direction of movement of the user, elevation of location of the user, etc. In some embodiments, the controller 110 may receive non-biometric data only. In some embodiments, the controller 110 may receive non-biometric data in addition to the biometric data. In some embodiments, the controller 110 may receive non-biometric data, biometric data, and past history of the user's behavior. For example, the controller 110 may receive non-biometric, biometric data, and past history of blood pressure of the user.

In some embodiments, the measured blood pressure is compared to a second predetermined threshold. The second predetermined threshold may be a lower limit for the blood pressure, e.g., 50 mm Hg, 60 mm Hg, 70 mm Hg, 80 mm Hg, 90 mm Hg, 100 mm Hg, 110 mm Hg, or 120 mm Hg. Determination of the second predetermined threshold can be based on the user's health condition, age, gender, etc. The second predetermined threshold can be changed at any time and added to the controller 110 to re-program the controller 110. Upon determination that the measured blood pressure is below the second predetermined threshold, the controller 110 transmits a signal to the pumping device 114 to activate the pumping device 114 and the compression unit 116. Subsequently, the pumping device 114/compression unit 116 pressurize the compression garment, i.e., the inflatable binder, to increase the blood pressure of the user. In some embodiments, the inflatable binder includes constricting materials, fibers or bands.

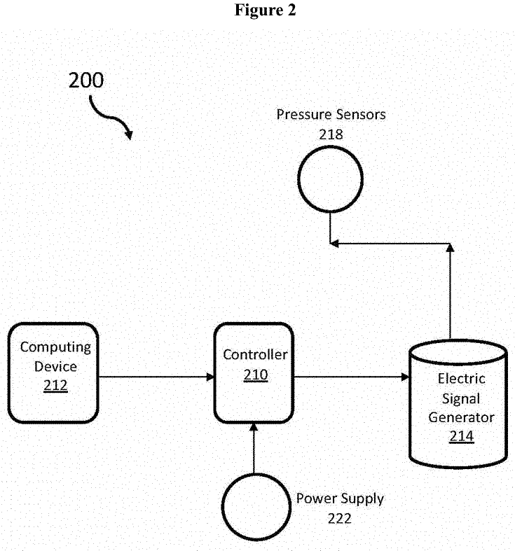

FIG. 2 illustrates the servo-controlled splanchnic venous compression with inflatable automated binder system 200 according to some embodiments. In some embodiments, the servo-controlled splanchnic venous compression with inflatable automated binder system 200 includes a controller 210 and a computing device 212 in communication with the controller 210, according to some embodiments. In some embodiments, the servo-controlled splanchnic venous compression with inflatable automated binder system 200 includes an electric signal generator 214, one or more pressure sensors 218 and a power supply 222. The electric signal generator 214 is in communication with the controller 210 and the one or more pressure sensors 218. The one or more pressure sensors 218 are further in contact with a user, e.g., a patient suffering OH or a pilot, to detect biometric data from the user and transmit the biometric data to the controller 210. The supply power 222 may be a rechargeable battery, a DC source of power, an AC source of power, or any other suitable source of power. The power supply 222 provides continuous operation of the controller 210.

In some embodiments, the controller 210 receives cardiovascular data from the user. The controller 210 may receive biometric data through the one or more pressure sensors 218. The biometric data may be blood pressure of the user. In some embodiments, the measured blood pressure is compared to a first predetermined threshold. The first predetermined threshold may be an upper limit for the blood pressure, e.g., 100 mm Hg, 110 mm Hg, 120 mm Hg, 130 mm Hg, 140 mm Hg, 150 mm Hg, 160 mm Hg, 170 mm Hg, 180 mm Hg, 190 mm Hg, or 200 mm Hg. Determination of the first predetermined threshold can be based on the user's health condition, age, gender, etc. The first predetermined threshold can be changed at any time and added to the controller 210 to re-program the controller 210. Upon determination that the measured blood pressure is above the first predetermined threshold, a warning is sent to the user. In some embodiments the warning is sent to at least one of: the user, a physician in charge of the user, and a third-party. Upon sending the warning, the controller 210 stops the process and the binder releases.

In some embodiments, the measured blood pressure is compared to a second predetermined threshold. The second predetermined threshold may be a lower limit for the blood pressure, e.g., 50 mm Hg, 60 mm Hg, 70 mm Hg, 80 mm Hg, 90 mm Hg, 100 mm Hg, 110 mm Hg, or 120 mm Hg. Determination of the second predetermined threshold can be based on the user's health condition, age, gender, etc. The second predetermined threshold can be changed at any time and added to the controller 210 to re-program the controller 110. Upon determination that the measured blood pressure is below the second predetermined threshold, the controller 110 transmits a signal to the electric signal generator 114. Subsequently, the electric signal generator 114 transmits an electric current to pressurize the compression garment, i.e., the inflatable binder, to increase the blood pressure of the user. In some embodiments, the inflatable binder includes constricting materials, fibers, or bands.

In some embodiments, the inflatable binder is an inflatable abdominal binder to be used around user's abdomen. In some embodiments, the inflatable binder is only activated when the user is standing. In some embodiments, the controller upon determination of a blood pressure below the second predetermined threshold, automatically commands the inflatable binder to inflate. The controller may be programmed to measure the blood pressure at certain times where the blood pressure was previously determined below the second predetermined threshold. For example, when the blood pressure falls below 100-60 at 6 am one day, the controller automatically measures the blood pressure next day at 6 am. The controller may be programmed to automatically repeat the measurement for a certain duration of time, e.g., a week, a month, etc.

The servo-controlled splanchnic venous compression with inflatable automated binder system measures the user's blood pressure intermittently and at predetermined times. In some embodiments, the controller is programmable to operate at certain times. For example, the controller is programmed to measure the user's blood pressure more frequently in early morning, when the user usually wakes up. The controller may further be programmed to measure the user's blood pressure less frequently during the night while the user is usually asleep.

In some embodiments, the servo-controlled splanchnic venous compression with inflatable automated binder system may include one or more additional sensors to detect user's biometric data other than the blood pressure, as a second biometric factor. This second biometric factor may be used to determine the user's current physical state, e.g., whether the user is asleep, awake, standing, walking, etc. For example, a heart rate sensor may be used to measure user's heart rate and compare it with rest heart rate or user's average heart rate for past certain period of time. Upon determination that the second biometric factor deviates from a presumed value, the controller may activate the process of inflating the binder.

In some embodiments, a machine learning model is used to program the controller. A suitable machine learning model may be initially trained by a suitable method. The trained machine learning model may then use ongoing data of the user's blood pressure for further training and anticipate user's blood pressure in the future. In some embodiments, the machine learning model can determine whether or not the user is standing. The machine learning model may measure the blood pressure at certain times where the blood pressure was previously determined below the second predetermined threshold.

In some embodiments, the machine learning model uses user's low blood pressure times to predict when the controller should measure the blood pressure more frequently. Alternatively, the machine learning model uses user's normal or relatively high blood pressure times to predict when the controller should measure the blood pressure less frequently.

In some embodiments, the servo-controlled splanchnic venous compression with inflatable automated binder system includes a machine learning model and one or more additional sensors. The machine learning model may use additional biometric data or other user data detected by the one or more additional sensors to predict required frequency of blood pressure measurements at different times of the day. By way of non-limiting examples, the one or more sensors may be an accelerometer, a gyroscope, a photosensor, a heart-rate sensor, an electrocardiograph sensor, a respiration sensor, an oxygen sensor, a glucose sensor, a pressure sensor, a temperature sensor, and/or a multi-variable sensor.

In some embodiments, the controller activates the measurement upon pressing a button by the user. In some embodiments, the controller measures the blood pressure while the inflation process is taking place.

In some embodiments, the machine learning model predicts when to measure the user's blood pressure more frequently in early mornings based on the history of the user. Alternatively, the machine learning model can predict when to measure the user's blood pressure less frequently during the night and while the user is asleep based on the user's behavior in going to bed.

One aspect of the subject matter is a method for treating a patient with orthostatic hypotension, comprising: determining a first and a second predetermined thresholds for the patient; equipping the patient with a servo-controlled splanchnic venous compression with inflatable automated binder system; collecting a biometric data of the patient from the one or more sensors; programming the binder system to automatically command the binder to contract upon determining the biometric data is below the second predetermined threshold; and programming the binder system to automatically command the binder to release upon determining the biometric data is at or above the first predetermined threshold. In some embodiments, the binder system includes a controller and a computing device in communication with the controller. In some embodiments, the system further includes a pumping device, a compression unit, one or more sensors, a valve, and a power supply. The pumping device may be in communication with the controller and the compression unit. In other embodiments, the binder system includes controller, a computing device in communication with the controller, an electric signal generator, one or more sensors, and a power supply. The electric signal generator is in communication with the controller and the one or more sensors. In various embodiments, the one or more sensors may be in contact with a user to detect biometric data from the user and transmit the biometric data to the controller. The biometric data may be blood pressure of the user measured by the one or more sensors. In some embodiments, the measured blood pressure is compared to a first and a second predetermined thresholds. The first predetermined threshold may be an upper limit for the blood pressure. Determination of the first and the second predetermined thresholds can be based on the user's health condition, age, gender, etc. The first and the second predetermined thresholds can be changed at any time and added to the controller to reprogram the controller.

Another aspect of the subject matter is a method for monitoring a user, the method being performed by a servo-controlled splanchnic venous compression with inflatable automated binder system. In some embodiments, the system includes a controller and a computing device in communication with the controller. In some embodiments, the system further includes a pumping device, a compression unit, one or more sensors, a valve, and a power supply. The pumping device may be in communication with the controller and the compression unit. In other embodiments, the system includes controller, a computing device in communication with the controller, an electric signal generator, one or more sensors, and a power supply. The electric signal generator is in communication with the controller and the one or more sensors. In various embodiments, the one or more sensors may be in contact with a user to detect biometric data from the user and transmit the biometric data to the controller.

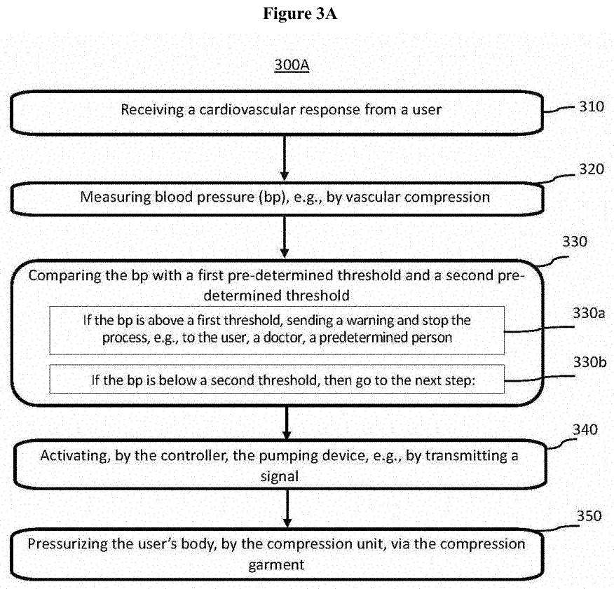

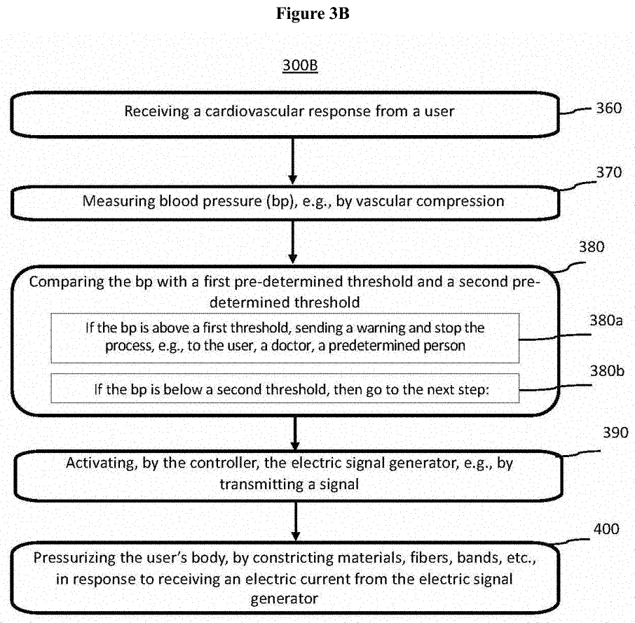

FIG. 3A is a flowchart representation of a method 300A of servo-controlled splanchnic venous compression with inflatable automated binder, in accordance with some embodiments. As represented by block 310, the method 300A includes receiving a cardiovascular response from a user. The user may be a pilot, a patient suffering OH, an elderly person, etc. As represented by block 320, the method includes measuring blood pressure. The blood pressure may be measured by vascular compression techniques or any other suitable technique. As represented by block 330, the method 300A includes comparing the blood pressure with a first predetermined threshold and a second predetermined threshold. The first predetermined threshold is used as an upper limit for the blood pressure. Upon determination that the measured blood pressure is above the first predetermined threshold, a warning is sent to at least one of: the user, a physician in charge of the user, and a third-party. Upon sending the warning, the controller stops the process, as represented by block 330A. The second predetermined threshold may be a lower limit for the blood pressure. Determination of the first and second predetermined threshold can be based on the user's health condition, age, gender, etc. Upon determination that the measured blood pressure is below the second predetermined threshold, i.e., block 330B, the controller transmits a signal to the pumping device to activate the pumping device and the compression unit, as represented by block 340. In some embodiments, the method 300A includes the pumping device/compression unit pressurizing the compression garment to increase the blood pressure of the user, as represented by block 350.

FIG. 3B is a flowchart representation of a method 300B of servo-controlled splanchnic venous compression with inflatable automated binder, in accordance with some embodiments. As represented by block 360, the method 300B includes receiving a cardiovascular response from a user. The user may be a pilot, a patient suffering OH, an elderly person, etc. As represented by block 370, the method 300B includes measuring blood pressure. The blood pressure may be measured by vascular compression techniques or any other suitable technique. As represented by block 380, the method 300B includes comparing the blood pressure with a first predetermined threshold and a second predetermined threshold. The first predetermined threshold is used as an upper limit for the blood pressure. Upon determination that the measured blood pressure is above the first predetermined threshold, a warning is sent to at least one of: the user, a physician in charge of the user, and a third-party. Upon sending the warning, the controller stops the process, as represented by block 380A. The second predetermined threshold may be a lower limit for the blood pressure. Determination of the first and second predetermined threshold can be based on the user's health condition, age, gender, etc. Upon determination that the measured blood pressure is below the second predetermined threshold, i.e., block 380B, the controller transmits a signal to the electric signal generator to activate the electric signal generator, as represented by block 390. In some embodiments, the method 300B includes transmitting, by the electric signal generator, an electric current to pressurize the compression garment, e.g., constricting materials, fibers or bands, to increase the blood pressure of the user, as represented by block 400.

One aspect of the subject matter disclosed herein is a compression garment, i.e., a splanchnic venous compression with binder system. The compression garment regulates venous splanchnic circulation. In some embodiments, the compression garment further includes a pumping device, a compression unit, a valve, one or more sensors, and a power supply. The pumping device may be in communication with the controller and the compression unit. In other embodiments, the compression garment includes controller, a computing device in communication with the controller, an electric signal generator, one or more sensors, and a power supply. The electric signal generator is in communication with the controller and the one or more sensors. In various embodiments, the one or more sensors may be in contact with a user to detect biometric data from the user and transmit the biometric data to the controller. The biometric data may be blood pressure of the user measured by the one or more sensors. In some embodiments, the measured blood pressure is compared to a first and a second predetermined thresholds. The first predetermined threshold may be an upper limit for the blood pressure. Determination of the first and the second predetermined thresholds can be based on the user's health condition, age, gender, etc. The first and the second predetermined thresholds can be changed at any time and added to the controller to reprogram the controller.

In some embodiments, the biometric data is transmitted over the Internet. Non-limiting examples of web-based transfer protocols include TCP/IP (Transmission Control Protocol/Internet Protocol), UDP/IP (User Datagram Protocol/Internet Protocol), HTTP (HyperText Transfer Protocol) and FTP (File Transfer Protocol). In some embodiments, the biometric data is sent to at least one of: the user, a physician in charge of the user, and a third-party. In some embodiments, the biometric data is stored in databases. In some embodiments, the biometric data is accessed through a digital processing device, a non-transitory computer readable storage medium, a computer program, a web application, a mobile application, a standalone plugin, or a web browser plug-in.

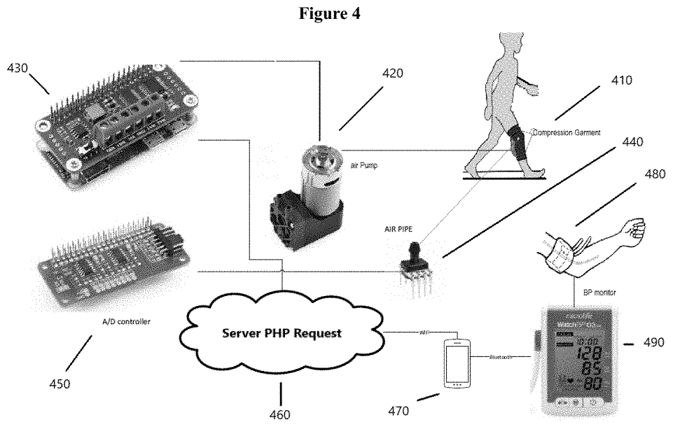

FIG. 4 illustrates a user wearing a compression garment 410, in accordance with some embodiments. In some embodiments, the compression garment 410 includes a pumping device 420 and a controller 430. The pumping device 420 is in communication with the controller 430. The compression garment 410 further includes one or more sensors 440. The one or more sensors 440 collect biometric data which is processed by an analog-to-digital converter 450. The biometric data is stored on a database 460. The database 460 is accessed from a digital processing device 470, e.g., a mobile phone, via an application, e.g., a computer program, a web application, a mobile application, a standalone application, and/or a web browser plug-in. The digital processing device 470 is in communication with the controller 430 and the database 460. In some embodiments, the compression garment further includes an adjustable cuff 480 with one or more sensors. The sensors collect biometric data, e.g., blood pressure data, which is sent to a blood pressure monitor 490. The monitor 490 is in communication with the digital processing device 470.

In some embodiments, the controller 430 receives biometric data from the user. The controller 430 and signals the pumping device 420 to inflate or deflate the compression garment 410. In some embodiments, the controller 430 receives the blood pressure data from the blood pressure monitor 490. In some embodiments, the blood pressure is compared to a first and a second predetermined thresholds. The first predetermined threshold may be an upper limit for the blood pressure. Determination of the first and the second predetermined thresholds can be based on the user's health condition, age, gender, etc. The first and the second predetermined thresholds can be changed at any time and added to the controller 430 to reprogram the controller 430.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

Although the subject matter has been described in language specific to structural features or methodological acts, it is to be understood that the subject matter of the appended claims is not necessarily limited to the specific features or acts described above. Rather, the specific features and acts described above are disclosed as example forms of implementing some of the embodiments.

Various operations of embodiments are provided herein. The order in which some or all of the operations are described should not be construed to imply that these operations are necessarily order dependent. Alternative ordering will be appreciated having the benefit of this description. Further, it will be understood that not all operations are necessarily present in each embodiment provided herein. Also, it will be understood that not all operations are necessary in some embodiments.

It will be appreciated that layers, features, elements, etc. depicted herein are illustrated with particular dimensions relative to one another, such as structural dimensions or orientations, for example, for purposes of simplicity and ease of understanding and that actual dimensions of the same differ substantially from that illustrated herein, in some embodiments.

Also, although the disclosure has been shown and described with respect to one or more implementations, equivalent alterations and modifications will occur to others of ordinary skill in the art based upon a reading and understanding of this specification and the annexed drawings. The disclosure comprises all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

### Certain Definitions

Unless otherwise defined, all technical terms used herein have the same meaning as commonly understood by one of ordinary skill in the art to which this invention belongs. “Exemplary” is used herein to mean serving as an example, instance, illustration, etc., and not necessarily as advantageous. As used in this application, “or” is intended to mean an inclusive “or” rather than an exclusive “or”. In addition, “a” and “an” as used in this application and the appended claims are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, or variants thereof are used, such terms are intended to be inclusive in a manner similar to the term “comprising”. Also, unless specified otherwise, “first,” “second,” or the like are not intended to imply a temporal aspect, a spatial aspect, an ordering, etc. Rather, such terms are merely used as identifiers, names, etc. for features, elements, items, etc. For example, a first element and a second element generally correspond to element A and element B or two different or two identical elements or the same element.

### Controller

In some embodiments, the devices, systems, and methods disclosed herein include at least one controller, or use of the same. A “controller” represents a chip, an expansion card, or a stand-alone device that interfaces with the garment, i.e., the servo-controlled splanchnic venous compression with inflatable automated binder in accordance with various embodiments. In some embodiments, the controller is in communication with a computing device. In other embodiments, the controller is in communication with an inflatable binder. In further embodiments, the controller is programmable by the user. In still further embodiments, the controller is programmable by feedback data generated by the devices, systems, and methods described in the subject matter. Those of skill in the art will recognize that various suitable controller manufacturers include, but are not limited to, Banana Pi, ClockworkPi, Orange Pi, Raspberry Pi®, Arduino®, ASUS™, BeagleBoard®, FriendlyARM, Huawei™, Khadas™, LattePanda®, LoveRPI®, Micro:Bit®, MinnowBoard, Nvidia™, Odroid, Onion, Pine64®, and UDOO®.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetB SD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux', and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony®PS3®, Sony®PS4®, Microsoft® Xbox 360®, Microsoft® Xbox One®, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for WebOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of virtual reality information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### EXAMPLES

The following illustrative examples are representative of embodiments of the devices, systems, and methods described herein and are not meant to be limiting in any way.

### Example 1

An elderly man experiences frequent syncopal symptoms when standing up after sitting for an extended period of time. He performs some research and concludes that his dizziness is likely a result of orthostatic hypotension. He decides to use a compressible garment to relieve his symptoms, which he adjusts to wrap around his abdomen. The compressible garment allows the man to enter in his vital statistics and program in threshold values for blood pressure and blood oxygen. The man sets a lower blood pressure threshold of 70 mm Hg, a lower blood oxygen threshold of 60 mm Hg, an upper blood pressure threshold of 110 mm Hg, and an upper blood oxygen threshold of 100 mm Hg. The compressible garment contains sensors that continually monitor the man's blood pressure and blood oxygen every second. The man stands up and feels dizzy, and the sensors detect that his blood pressure is presently 69 mm Hg. The compressible garment contracts around the man's abdomen and his blood pressure is raised. The sensors now detect the man's blood pressure at 110 mm Hg, and the compressible garment stops contracting and releases, thereby stopping the process.

### Example 2

An Air Force fighter pilot is undergoing dogfighting training when she banks her airplane hard to the right and experiences over 2 g-forces of lateral acceleration. Her compression device detects her blood oxygen to be dropping below 70 mm Hg, her preset minimum. The compression device signals a connected oxygen tank to release oxygen into the pilot's flight suit to assist in her recovery.

### Example 3

A teenager experiences spells of dizziness and does not know the cause. Her physician suggests that she use a venous compression system to monitor her blood pressure. The venous compression system appears to be a finely crafted cloth band woven out of fine metal strands. The sensors of the venous compression system continually monitor the teenager's blood pressure. The sensors also track her daily activity, including when and how often she sleeps, walks, sits, and stands up from a sitting position. The data from the sensors is compiled and sent wirelessly directly to her physician. Based on the data, the physician makes a determination that the teenager likely suffers from orthostatic intolerance. The physician informs her that certain actions may worsen her spells of dizziness. Based on the teenager's daily activity tracked by the sensors, the physician helps the teenager understand when she is most at risk of being dizzy and advises her on changes she should make to her daily routines.

What is claimed is:

1. A compressible garment comprising: a servo-controlled splanchnic venous compression with automated binder system comprising: a controller, a computing device in communication with the controller, a binder, one or more sensors, and a power supply.

2. The compressible garment of claim 1, wherein the controller is programmable by a user.

3. The compressible garment of claim 1, wherein the binder is adjustable around a user's body.

4. The compressible garment of claim 1, wherein the binder is activated manually by a user.

5. The compressible garment of claim 1, wherein the binder comprises a pumping device and a compression unit.

6. The compressible garment of claim 1, wherein the binder comprises an electric signal generator. The compressible garment of claim 6, wherein the binder further comprises a contractible fabric, wherein the contractible fabric contracts upon a stimulus by the electric signal generator.

8. The compressible garment of claim 6, wherein the electric signal generator generates an electric current to cause a pulling of a wire to contract the binder.

9. The compressible garment of claim 1, wherein the one or more sensors collect biometric data from a user.

10. The compressible garment of claim 9, wherein the biometric data is used to determine a physical state of the user.

11. The compressible garment of claim 9, wherein the controller uses the biometric data collected from the user to self-program through a machine learning model.

12. The compressible garment of claim 9, wherein the biometric data is blood pressure.

13. The compressible garment of claim 12, wherein the blood pressure is compared to a first predetermined threshold and a second predetermined threshold.

14. The compressible garment of claim 13, wherein the controller, upon determining the blood pressure is above the first predetermined threshold, a warning is issued.

15. The compressible garment of claim 13, wherein the controller, upon determining the blood pressure is at or above the first predetermined threshold, automatically commands the binder to release.

16. The compressible garment of claim 13, wherein the controller, upon determining the blood pressure is below the second predetermined threshold, automatically commands the binder to contract.

17. The compressible garment of claim 14, wherein the warning is issued to at least one of: the user, a physician, and a third-party.

18. The compressible garment of claim 14, wherein the warning is issued through an ancillary communication system.

19. A venous compression device comprising: a controller, a computing device in communication with the controller, a binder, one or more sensors, and a power supply.

20. A method for treating a patient with orthostatic hypotension, comprising:
a) determining a first and a second predetermined thresholds for the patient;
b) equipping the patient with a servo-controlled splanchnic venous compression with inflatable automated binder system comprising: a controller, a computing device in communication with the controller, a binder, one or more sensors, and a power supply;
c) collecting a biometric data of the patient from the one or more sensors;
d) programming the controller to automatically command the binder to contract upon determining the biometric data is below the second predetermined threshold; and
e) programming the controller to automatically command the binder to release upon determining the biometric data is at or above the first predetermined threshold.
